(19) United States
(12) Patent Application Publication
(10) Pub. No.: US 2021/0338601 A1
(43) Pub. Date: Nov. 4, 2021

(54) ENHANCED PURITY AND METHODS OF USE OF ABLATIVE ALCOHOL

(71) Applicant: Belcher Pharmaceuticals, LLC, Largo, FL (US)

(72) Inventor: Darren Rubin, Largo, FL (US)

(21) Appl. No.: 17/372,753

(22) Filed: Jul. 12, 2021

Related U.S. Application Data
(62) Division of application No. 15/933,631, filed on Mar. 23, 2018, now abandoned.

(51) Int. Cl. A61K 31/045; A61M 25/10

(52) U.S. Cl. CPC A61K 31/045; A61M 25/104; A61M 2202/0484; A61M 2025/1056; A61M 2210/127; A61M 25/10182

(57) ABSTRACT

A liquid injectable pharmaceutical drug product consisting of no less that 99 percent ethanol comprised of no less than 99 percent ethanol active pharmaceutical ingredient with a volume of at least 0.05 mL to be injected into a patient as a method of ablating/lysing cells.

The present invention provides alcohol of enhanced purity for pharmaceutical use and improved methods for ablating tissues and performing therapeutic neurolysis.

### BACKGROUND OF THE INVENTION

Alcohols are the class of organic compounds containing a hydroxyl functional group (—OH) bound to a saturated carbon atom. While the term “alcohol” may refer to any member of this class, the term commonly refers to ethyl alcohol or ethanol having the chemical formula C2H5OH. Ethanol is perhaps one of the earliest medicinal substances known since Neolithic times, dating back to almost ten thousand years when mankind began fermenting sugary beverages into ethanol for its intoxicating effects. Millennia later, mankind learned the distillation process of how to concentrate fermented ethanol, to reduce, but not eliminate, its water content and impurities.

The present invention represents the first pharmaceutical drug product produced from synthetic ethanol that is absolute and essentially free of water and having a purity of at least 99 percent ethanol, and is therefore, more pure than other medicinal ethanol solutions.

The present invention also represents improved methods of injecting alcohol for pharmacological purposes, including methods of injecting ethanol having a purity of at least 99 percent for pharmacological purposes, including, but not limited to, tissue ablation, tumor ablation, and therapeutic neurolysis.

There exists a great need for a pharmaceutical grade alcohol drug product that solves the problems inherent in less pure, unapproved medicinal alcohol products having less than 99 percent ethanol; one that provides the assurance of quality and a low impurity profile; one that is preferably made from a pure, synthetic ethanol active pharmaceutical ingredient, and not from unclean grain alcohol fermentative processes. The present invention fulfills this need and provides the advantage of higher stability and reduced exposure to toxic impurities. The present invention and methods improves the standard of patient care, even for rare diseases. Patients include humans, but can also include veterinary patients.

### DETAILED DESCRIPTION OF THE INVENTION

The present invention is a pharmaceutical drug product containing at least 99 percent pure ethanol, made from at least 99 percent pure ethanol active pharmaceutical ingredient. In preferred embodiments, the drug product is contained in an ampoule and or vial and or bottle. In other embodiments, the drug product is contained in a catheter and or prefilled syringe. In preferred embodiments, the drug product is made from synthetic ethanol active pharmaceutical ingredient and not from fermented ethanol.

Chemical synthesis of pure ethanol for the purposes of the active pharmaceutical ingredient and drug product according to this invention is preferably derived from ethylene produced from the petrochemical industry during “steam cracking” of hydrocarbons to produce lighter alkenes. Ethylene is converted to ethanol, preferably by acid-catalyzed hydration, although other methods are available, such as with a diethyl sulfate intermediate or other redox reactions. Ethanol may be further purified by fractional distillation and further filtered to guarantee quality of the active pharmaceutical ingredient and or drug product for injection.

The invention is a pharmaceutical drug product of no less than 99 percent ethanol for injection. The invention is a pharmaceutical drug product of no less than 99 percent synthetic ethanol for injection. The invention is a pharmaceutical drug product of no less than 99 percent ethanol for injection contained in a glass and or plastic container holding at least 0.05 mL of this drug product, and preferably 1 mL to 5 mL of this drug product, the glass and or plastic container selected from a glass and or plastic ampoule, a glass and or plastic vial, a glass and or plastic prefilled syringe. In other embodiments, the drug product is used with or contained by a catheter containing at least 1 mL, and preferably up to 10 mL, of this drug product. One preferred catheter is a percutaneous transluminal catheter, or over-the-wire percutaneous transluminal coronary angioplasty balloon catheter, containing this drug product. In other embodiments, the invention is a reusable, multiple-dose vial or bottle preferably containing 5 mL or more of this drug product. In most preferred embodiments, the shelf-life of the product is at least 12 months, and preferably at least 48 months long.

The injectable ethanol drug product according to this invention is used for tissue ablation and or therapeutic neurolysis because pure ethanol is highly lytic to mammalian cells. The ethanol drug product causes direct damage to nerve cells via dehydration, necrosis of cells, and leaking of protoplasm, leading to neuritis and a pattern of anterograde degeneration. However, it is important to have ethanol of the highest purity to ensure lysis with as minimum a volume as possible so that nearby healthy tissue is not affected/not affected as much by the ethanol itself, and so that healthy tissue is not exposed to elemental or chemical toxic impurities that would otherwise be found in unapproved medicinal ethanol products. Pure ethanol is necrotic to both healthy and diseased tissue. Some methods according to the invention include tumor ablation with this drug product according to this invention, and may include head and neck tumors and liver tumors ablation. In other methods according to the invention, this drug product ablates when injected directly into infected, ischemic, ulcerative, or cirrhotic tissue. In some instances, an intralesional injection of ethanol is required. In other instances, injection into a vein, artery, and or arteriole of a problematic organ is necessary so that the ethanol perfuses into that organ for ablation (e.g., such as for ablating hepatocellular carcinoma).

When this drug product according to the invention is injected into or adjacent to nerves or ganglia, enhanced neurolysis occurs, which is useful for treating intractable pain associated with cancer or a terminal illness, and even for treating trigeminal neuralgia.

The invention also includes methods of performing Percutaneous Transluminal Septal Myocardial Ablation (alcohol or ethanol septal ablation). Inventive methods of injecting the alcohol drug product into septal arterial branch(es) according to this invention include inducing controlled cardiac septal infarction to improve exercise capacity in patients with symptomatic hypertrophic obstructive cardiomyopathy who are not candidates for surgical myectomy and who are often drug-refractory. For individuals with enlarged, hypertrophic septum walls of their heart (e.g., 15 mm or greater) the current invention offers an alternative to surgical myectomy, especially for sick or elderly patients that may not survive the thoracotomy and cardiopulmonary bypass that surgical myectomy involves. Such patients have hypertrophic obstructive cardiomyopathy, an often genetic condition, with a high left ventricular outflow tract pressure gradient (e.g., 50 mmHg or greater), which this procedure alleviates along with alleviating dyspnea, angina, and improves exercise capacity in these individuals. The septum size is reduced with this method following a localized infarction caused by the drug product according to this invention.

In preferred embodiments, the methods use a percutaneous transluminal catheter, or over-the-wire percutaneous transluminal coronary angioplasty balloon catheter, the lumen of which delivers contrast agent and the at least 99 percent ethanol drug product of this invention into the target septal artery. Generally, a temporary pacing wire is inserted as transient heart block is common with this method. A guiding catheter is introduced into the left main coronary artery and a guidewire (e.g., about 0.014 inch diameter) into the left anterior descending artery. A short (e.g., about 10 mm long, about 1.5 to 2.5 mm diameter) over the wire balloon is placed into the septal artery branch under at least one type of guidance selected from the class of cardiac guidance that includes contrast echocardiographic guidance, fluoroscopic guidance, pressure gradient-fluoroscopic guidance, cardiac magnetic resonance imaging guidance, and contrast-enhanced magnetic resonance imaging guidance, some of which use intracoronary contrast injection. After inflation of the balloon, the guidewire is removed. To ensure the alcohol will not spill into the left anterior descending artery, injection of angiographic contrast agent through the balloon can be done. In most embodiments, injection of echocardiographic contrast agent via the balloon catheter helps to verify that the region to be infarcted is the portion of the septum responsible for the obstruction in the left ventricular outflow tract, before actually infarcting that region with ethanol. In some embodiments of this method, a transient reduction in left ventricular outflow tract pressure gradient can be tested prior to the alcohol injection by means of transient balloon occlusion. This allows assessment of the gradient. Once the desired septal artery is determined, at least 99 percent pure ethanol drug product according to this invention is preferably slowly injected as a small volume, 0.5 mL to 5 mL, and preferably 1 mL to 2 mL, and even more preferably 1.4 mL to 1.5 mL, or about 1.45 mL, is injected by the percutaneous transluminal catheter, or occlusion balloon catheter, over the course of preferably 1 to 2 minutes into each target septal artery to be ablated. Ideally, the balloon should remain inflated in place in the septal artery for several minutes, and preferably for about 5 minutes, to prevent ethanol spillage/spreading into the patient's bloodstream. After injection of the ethanol drug product, the balloon and wire are removed. In some instances, it may be desired to inject the ethanol drug product into a second or additional septal artery during the same procedure or a subsequent procedure. Generally, the methods use the minimal dose necessary to achieve the desired reduction in peak left ventricular outflow tract pressure gradient.

Again, the proximal septal branch of the left anterior descending artery that supplies blood to the hypertrophied septum is what is cannulated and receives the ethanol according to this invention. Inside this injected vessel, blood becomes lumpily fixed by the ethanol, and the ethanol is trapped and absorbed in this necrotic zone. This occludes the vessel with a chemically induced myocardial infarction of the basal septum, causing a decrease in septal thickness. This is accompanied by scar formation and remodeling over time (e.g., several weeks or more) of the left ventricular outflow tract and a reduction in its gradient pressure.

It is desirable for the left ventricular outflow tract pressure gradient to decrease below 20 mmHg, and more preferably below 16 mmHg, with this procedure and for about a 6 mm or more reduction in septal wall thickness in the troubled area to below 15 mm in adults. Proper identification of the optimal septal vessel for ablation is important for achieving this goal which is accompanied by decreased angina, decreased dyspnea, and an increase in exercise capacity.

A primary embodiment of the invention is a liquid injectable pharmaceutical drug product consisting of no less than 99 percent ethanol comprised of no less than 99 percent ethanol active pharmaceutical ingredient with a volume of at least 0.05 mL to be injected into a patient as a method of ablating/lysing cells. In preferred embodiments, the no less than 99 percent ethanol active pharmaceutical ingredient consists of fully synthetic, non-fermented ethanol and the drug product has a shelf-life of at least 12 months. In preferred embodiments, this liquid injectable pharmaceutical drug product is held and or stored in an at least one pharmaceutically acceptable container selected from containers including ampoules, vials, bottles, catheters, and syringes; the at least one pharmaceutically acceptable container is comprised of glass, polymer, or a combination thereof. In a some preferred embodiments, this liquid injectable pharmaceutical drug product is held in a pharmaceutically acceptable container associated with a percutaneous catheter, or the percutaneous catheter itself as a drug device combination. In other embodiments, the percutaneous catheter and the at least one pharmaceutically acceptable container holding the liquid injectable pharmaceutical drug product are provided together as a kit.

The above examples and measurement examples are not meant to be limiting.

In many primary embodiments, this liquid injectable pharmaceutical drug product is injected into a blood vessel of the patient, to ablate that blood vessel, such as a varicose vein. However, in most instances, this liquid injectable pharmaceutical drug product is injected into a blood vessel of, or supplying blood to, a diseased/problematic organ of the patient.

In other embodiments, this liquid injectable pharmaceutical drug product is injected into a lymphatic vessel or lymph node of the patient.

In still other embodiments, this liquid injectable pharmaceutical drug product is injected into a tumor of the patient.

In yet still other embodiments, this liquid injectable pharmaceutical drug product is injected into diseased/problematic tissue of the patient. This may include direct injection into organs such as the brain, kidney, pancreas, liver, lung, prostate, sexual reproductive tissue, or a combination thereof in the patient, to treat a disease, for example, if tumors were present.

In yet still other embodiments, this liquid injectable pharmaceutical drug product has veterinary uses, such as a nonsurgical alternative to spaying and neutering with direct chemical ablation.

In some embodiments, the liquid injectable pharmaceutical drug product is injected into a cyst of the patient. A cyst may include an ovarian cyst.

In some embodiments, the liquid injectable pharmaceutical drug product is injected into inflamed/infected tissue of the patient.

In some embodiments, the liquid injectable pharmaceutical drug product is injected into ischemic tissue of the patient.

In some embodiments, the liquid injectable pharmaceutical drug product is injected into ulcerative tissue of said patient. In some cases, ulcerative tissue can be caused by diabetes or gastrointestinal problems.

In some embodiments, the liquid injectable pharmaceutical drug product is injected into a cirrhotic tissue/fibrosis of the patient. Cirrhotic tissue may include that of a diseased liver.

In some embodiments, the liquid injectable pharmaceutical drug product is injected into and or along a nerve and or ganglion of the patient.

The invention includes methods of using this liquid injectable pharmaceutical drug product consisting of no less than 99 percent ethanol comprised of no less than 99 percent ethanol active pharmaceutical ingredient with a volume of at least 0.05 mL to be injected into a patient as a method of ablating/lysing cells.

The invention includes methods of lysing an at least one nerve/ganglion by injecting this liquid injectable pharmaceutical drug product into an at least one nerve/ganglion.

The invention includes methods of lysing an at least one blood vessel by injecting this liquid injectable pharmaceutical drug product into an at least one blood vessel.

The invention includes methods of causing an infarction of an at least one organ tissue by injecting said liquid injectable pharmaceutical drug product into an at least one blood vessel.

The invention includes methods of ablating an at least one organ tissue by causing an infarction of an at least one organ tissue by injecting this liquid injectable pharmaceutical drug into an at least one blood vessel.

The invention includes methods of ablating an at least one organ tissue by injecting and or perfusing this liquid injectable pharmaceutical drug product into said at least one organ tissue.

Primary embodiments of this invention includes methods of reducing septal thickness by at least 2 mm to reduce left ventricular outflow tract pressure gradient by at least 2 mmHg by injecting this liquid injectable pharmaceutical drug product into an at least one septal artery of a heart of a patient to cause a localized infarct therein.

One such primary embodiment of this invention includes a method of reducing septal thickness by at least 2 mm to reduce left ventricular outflow tract pressure gradient by at least 2 mmHg by injecting this liquid injectable pharmaceutical drug product consisting of no less than 99 percent ethanol comprised of no less than 99 percent ethanol active pharmaceutical ingredient, into an at least one target septal artery of a heart of a patient to cause a localized infarct therein. This method includes the step of measuring left ventricular outflow tract pressure gradient. This method further includes the step of placing a guiding catheter into the left main coronary artery and a guidewire into the left anterior descending artery. This method further includes placing an over-the-wire balloon of a percutaneous transluminal catheter or over-the-wire percutaneous transluminal coronary angioplasty balloon catheter into a septal artery branch under at least one type of guidance selected from the class of cardiac guidance that includes contrast echocardiographic guidance, fluoroscopic guidance, pressure gradient-fluoroscopic guidance, cardiac magnetic resonance imaging guidance, and contrast-enhanced magnetic resonance imaging guidance, or a combination thereof to help determine an at least one potential target septal artery. This method further includes the step of injecting an angiographic/echocardiographic contrast agent (or intracoronary contrast agent) through a central balloon lumen to ensure subsequent injection of this liquid injectable pharmaceutical drug product does not spill into the left anterior descending artery and to help verify that a heart region to be infarcted is the portion of the septum responsible for causing the obstruction in the left ventricular outflow tract before continuing to the next step. This method optionally includes transient occlusion of the septal artery with the over-the-wire balloon to test a transient reduction in left ventricular outflow tract pressure gradient prior to continuing to the next step. This method further includes the step of slowly injecting at least 0.5 mL and up to 5 mL, and preferably about 1.45 mL of this liquid injectable pharmaceutical drug product through an at least one lumen of the percutaneous transluminal catheter or over-the-wire percutaneous transluminal coronary angioplasty balloon catheter over the course of preferably 1 to 2 minutes into the target septal artery to be ablated and keeping said over-the-wire balloon inflated in this target septal artery for at least 2 minutes to prevent spillage and repeating this step if another target septal artery is to be ablated. This method further includes the step of removing the over-the-wire balloon and or percutaneous transluminal catheter or over-the-wire percutaneous transluminal coronary angioplasty balloon catheter and any remaining guidewire. This method further optionally and preferably includes the step of remeasuring the left ventricular outflow tract pressure gradient. In most embodiments, this method further includes the step of placement of a temporary pacing wire in the heart of the patient, when the patient does not already have a permanent pacemaker. This is done to help counter a potential transient heart block.

Measuring the left ventricular outflow tract pressure gradient is often performed by advancing a multipurpose catheter into the apex of the left ventricle and placing a guiding catheter in the aorta to determine the pressure gradient therebetween. A ventricular outflow tract is a portion of a ventricle of the heart through which blood passes in order to enter the great arteries. The left ventricular outflow tract pressure gradient may also be calculated or estimated by Doppler or echocardiographic or ultrasound means.

The invention also includes a percutaneous transluminal catheter/over-the-wire percutaneous transluminal coronary angioplasty balloon catheter associated with a pharmaceutically acceptable container holding this liquid injectable pharmaceutical drug product consisting of no less than 99 percent ethanol comprised of no less than 99 percent ethanol active pharmaceutical ingredient. This balloon catheter and balloon is preferably fully alcohol compatible with negligible leachables and negligible degradation of said catheter, even over a long shelf-life.

The invention includes methods of producing and manufacturing this drug product.

In still further embodiments, the no less than 99 percent ethanol active pharmaceutical ingredient is combined with one or more other active pharmaceutical ingredients and or one or more excipient ingredients. In some embodiments, one or more other alcohols or solvents (e.g., phenol) may be used with or instead of ethanol.

The above examples and measurement examples are not meant to be limiting. Choice, design, and sizes of container closure systems, syringe and or catheter devices, optional other potential ingredients, along with other variations and embodiments of the methods of the invention described herein will now be apparent to those of skill in the art without departing from the disclosure of the invention or the coverage of the claims to follow.

1. A method of reducing septal thickness in a patient, the method comprising:
measuring a left ventricular outflow tract pressure gradient;
placing a guiding catheter into a left main coronary artery and a guidewire into a left anterior descending artery;
placing an over-the-wire balloon of a percutaneous transluminal catheter or over-the-wire percutaneous transluminal coronary angioplasty balloon catheter into a septal artery branch under at least one type of guidance to determine an at least one potential target septal artery;
injecting an angiographic/echocardiographic contrast agent through a central balloon lumen to ensure subsequent injection of a liquid injectable pharmaceutical drug product does not spill into the left anterior descending artery and to verify that a heart region to be infarcted is a portion of a septum responsible for an obstruction in a left ventricular outflow tract;
slowly injecting at least 0.5 mL and up to 5 mL of the liquid injectable pharmaceutical drug product through an at least one lumen of the percutaneous transluminal catheter or over-the-wire percutaneous transluminal coronary angioplasty balloon catheter over the course of 1 to 2 minutes into the target septal artery and keeping the over-the-wire balloon inflated in the target septal artery for at least 2 minutes to prevent spillage; and
removing the over-the-wire balloon and the percutaneous transluminal catheter or over-the-wire percutaneous transluminal coronary angioplasty balloon catheter and any remaining guidewire,
wherein the method reduces septal thickness by at least 2 mm and reduces left ventricular outflow tract pressure gradient by at least 2 mmHg; and
wherein the liquid injectable pharmaceutical drug product consists of no less than 99 percent ethanol consisting of no less than 99 percent of fully synthetic, non-fermented ethanol; and.

2. The method of claim 1, further comprising transiently occluding the target septal artery with the over-the-wire balloon to test a transient reduction in left ventricular outflow tract pressure gradient prior to injecting the liquid injectable pharmaceutical drug product.

3. The method of claim 1, wherein about 1.45 mL of the liquid injectable pharmaceutical drug product is injected.

4. The method of claim 1, further comprising repeating the injecting in another target septal artery.

5. The method of claim 1, further comprising re-measuring the left ventricular outflow tract pressure gradient after removing the over-the-wire balloon and the percutaneous transluminal catheter or over-the-wire percutaneous transluminal coronary angioplasty balloon catheter and any remaining guidewire.

6. The method of claim 1, further comprising placing a temporary pacing wire in the heart of the patient, when the patient does not have a permanent pacemaker, to help counter a potential transient heart block.

7. The method of claim 1, wherein the at least one type of guidance is selected from contrast echocardiographic guidance, fluoroscopic guidance, pressure gradient-fluoroscopic guidance, cardiac magnetic resonance imaging guidance, and contrast-enhanced magnetic resonance imaging guidance, or a combination thereof.
